(19) United States
(12) Patent Application Publication
(10) Pub. No.: US 2026/0048160 A1
(43) Pub. Date: Feb. 19, 2026

(54) Violet-Blue Visible Light Sterilization Device

(71) Applicants: The Government of the United States, as Represented by the Secretary of the Army, Ft. Detrick, MD (US); The United States of America, as Represented by the Secretary of the Navy, Silver Spring, MD (US)

(72) Inventors: Bradley T. Pierce, Kailua, HI (US); Lisa M. Pierce, Kailua, HI (US); Jason B. Brill, Kaneohe, HI (US)

(21) Appl. No.: 19/370,885

(22) Filed: Oct. 28, 2025

Related U.S. Application Data
(63) Continuation-in-part of application No. PCT/US2024/027345, filed on May 2, 2024.
(60) Provisional application No. 63/463,954, filed on May 4, 2023.

(51) Int. Cl. A61L 2/08

(52) U.S. Cl. CPC A61L 2/084; A61L 2202/11; A61L 2202/14; A61L 2202/16; A61L 2202/24

(57) ABSTRACT

A sterilization device includes at least one flexible sheet; a plurality of violet-blue visible light light-emitting diodes (LEDs) at least partially or fully embedded in or attached to the at least one flexible sheet; and a housing having at least one reflective surface into which the at least one flexible sheet is insertable.

This US patent application is a Continuation-in-Part of PCT international application PCT/US2024/027345 filed on 2 May 2024, which claims priority to U.S. Ser. No. 63/463,954, filed in the US Patent and Trademark Office on 4 May 2023, the entireties of which are incorporated herein by reference.

### FIELD OF INVENTION

The present invention is directed to a sterilization device. In particular, the sterilization device may have a plurality of violet-blue visible light emitting diodes (LED) at least partially embedded in a flexible sheet and contained in a housing having at least one reflective surface.

### BACKGROUND OF INVENTION

Operational situations in which U.S. Special Operations Forces are deployed to remote and austere locations and combat scenarios involving future joint, multi-domain, and large-scale combat operations have resulted in a shift in focus toward prolonged field care.

Combat traumatic wound infections and delayed access to care in austere environments, where evacuation to higher levels of care is not immediately possible, is a major risk to morbidity and recovery and underscores the urgency for wound infection prevention. The unavailability of sterile surgical instruments in remote locations may impede the immediate surgical care that is critical for survival. If non-sterile surgical tools are used, serious complications may result, including increased surgical site infections, sepsis, and even death.

Thus, a requirement exists for portable sterilizers that can easily be transported to remote locations to provide sterile surgical instruments when needed. Currently, military surgical teams use heavy steam sterilizers that require significant power and potable water, making them impractical for field use, especially in environments where power generation and/or water are limited.

An innovative sterilization approach in the form of a lightweight, portable decontamination device is needed.

### SUMMARY OF INVENTION

The invention provides in a first embodiment a sterilization device comprising at least one flexible sheet; a plurality of violet-blue visible light light-emitting diodes (LEDs), each LED at least partially or fully embedded in or attached to the at least one flexible sheet; and a housing having at least one reflective surface. The sterilization device may include an array of violet-blue LEDs having a wavelength of about 400 to about 420 nm.

The invention provides in a second embodiment further to any of the previous embodiments a sterilization device comprising an array of violet-blue LEDs having a wavelength of about 405 nm.

The invention provides in a third embodiment further to any of the previous embodiments a sterilization device wherein the at least one flexible sheet comprises a silicone sheet. The silicone sheet may have a thickness of about 0.1 to about 1 mm.

The invention provides in a fourth embodiment further to any of the previous embodiments a sterilization device wherein the housing comprises a MYLAR® bag.

The invention provides in a fifth embodiment further to any of the previous embodiments a sterilization device comprising a circuit board structure having a plurality of metal strips, each strip attached at one end to a positive voltage supply rail and at an opposing end to negative ground power rail, wherein two or more violet-blue LEDs are affixed to each metal strip. The metal strips may comprise aluminum.

The invention provides in a sixth embodiment further to any of the previous embodiments a sterilization device wherein the violet-blue LEDs are connected via a wire mesh.

The invention provides in a seventh embodiment further to any of the previous embodiments a sterilization device wherein the plurality of metal strips is configured so that a back or rear surface of each strip is exposed to the air within the sterilization device.

The invention provides in an eighth embodiment further to any of the previous embodiments a sterilization device comprising at least one fan and at least one air vent.

The invention provides in a ninth embodiment further to any of the previous embodiments a sterilization device comprising at least one rechargeable battery.

The invention provides in a tenth embodiment further to any of the previous embodiments a sterilization device in which the at least one flexible sheet comprising the plurality of violet-blue visible light LEDs is folded into two layers and is configured so that a number of LEDs face each other and a space is formed between the two layers for an object to be sterilized.

The invention provides in an eleventh embodiment further to any of the previous embodiments a sterilization device further comprising a reflective or mirrored sheet or coating attached to the at least one flexible sheet.

The invention provides in a twelfth embodiment further to any of the previous embodiments a sterilization device in which the at least one flexible sheet has a plurality of plastic elements, with one violet-blue LED mounted on and/or attached to each plastic element.

The invention provides in a thirteenth embodiment further to any of the previous embodiments a sterilization device in which each LED has its own heat sink comprising a body having at least one projection or fin extending outwardly from the body. The heat sink may comprise two or more projections or fins extending outwardly from the body.

The invention provides in a first method embodiment a method comprising placing or wrapping an object in need of sterilization in a device according to any one of the previous embodiments; and applying violet-blue visible light from the violet-blue visible light LEDs to the object for a set amount of time, thereby sterilizing the object by reducing or eliminating at least one of bacteria or viruses on the object.

The invention provides in a second method embodiment further to any of the previous method embodiments a method wherein the object in need of sterilization comprises at least one surgical instrument.

An advantage of the sterilization device is that it is lightweight and portable. Thus, the device can be used in austere or resource-limited environments.

Another advantage of the sterilization device is that it is that it can be powered by a rechargeable battery or an external power source.

Another advantage of the sterilization device is that it has reduced energy requirements, long operational use, and low maintenance.

### BRIEF DESCRIPTION OF THE DRAWINGS

FIG. 1 is a perspective view of a violet-blue visible light sterilization device according to an embodiment of the present invention.

FIG. 2 is an exploded perspective view of the device shown in FIG. 1.

FIG. 3 is an exploded top view of the device shown in FIG. 1.

FIG. 4 is an exploded view of the LED array and connector of the device shown in FIG. 1.

FIG. 5 is a side partial cross-sectional view of a violet-blue visible light sterilization device with an exemplary surgical tool to be sterilized according to an embodiment of the present invention.

FIG. 6 is a top view of a flexible sheet according to another embodiment of the present invention.

FIG. 7 is a side partial cross-sectional view of a sterilization device according to another embodiment of the present invention.

### DETAILED DESCRIPTION OF INVENTION

The present invention is directed to a sterilization device comprising a plurality of violet-blue visible light emitting diodes (LED) at least partially or fully embedded in at least one flexible sheet and contained in a housing having at least one reflective surface. The device may be carried in a ruck sack, having the approximate size and weight of a combat MRE ration. In embodiments, the sterilization device may be about 1 lb. to about 5 lb.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

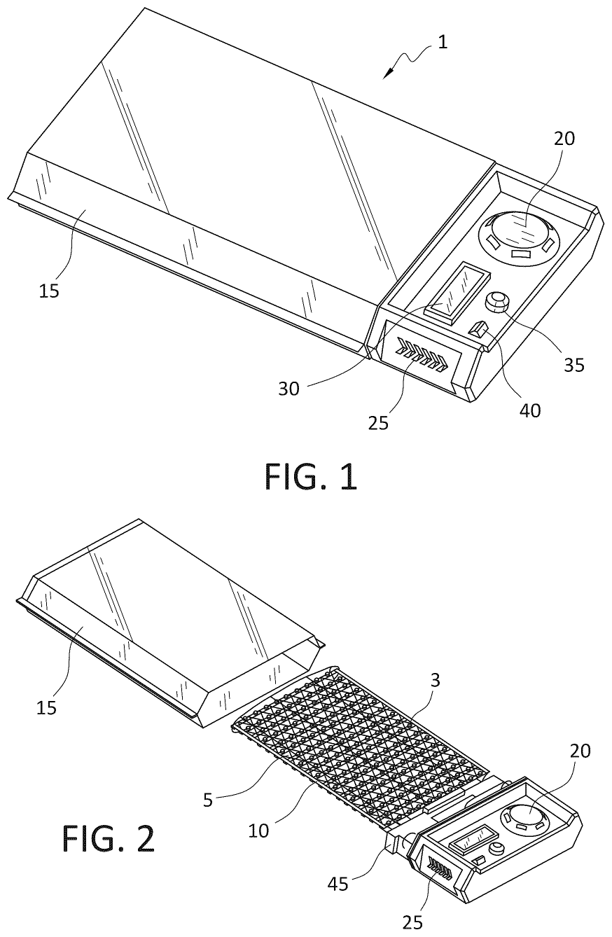

As shown in FIGS. 1-2, a sterilization device 1 according to the present invention comprises a plurality of violet-blue visible light emitting diode (LEDs) 3. In embodiments, the plurality of violet-blue LEDs may comprise an array 5 (FIG. 2). The number of violet-blue LEDs may be selected based upon at least one of a desired size of the device, the items to be sterilized, or the time of sterilization.

The violet-blue LEDs each emit light having a wavelength in a range of about 400 nm to about 420 nm. The violet-blue LEDs possess reduced energy requirements, long operational use, low maintenance, and lower photon energy compared to ultraviolet (UV) light and do not cause degradation of photosensitive materials, including various plastics and silicone.

In one embodiment, the violet-blue LEDs each emit visible light having a wavelength of about 405 nm. Non-limiting advantages of 405 nm violet-blue visible light technology may include, but are not limited to, efficacy against a broad range of multidrug-resistant Gram-positive and Gram-negative bacteria, fungi, yeasts, and viruses, and low risk of resistance development by pathogens.

The violet-blue LEDs 3 or array 5 may be at least partially or fully embedded in or attached to a flexible sheet 10. In an embodiment, the LEDs 3 or array 5 are fully embedded and fixed within the flexible sheet and are not removable. In an embodiment, the flexible sheet is a flexible silicone sheet, which may be transparent. In embodiments, the flexible sheet 10 may have a thickness of about 0.1 mm to about 1 mm, for example 0.1 mm to 0.5 mm.

The flexible sheet may be placed in a housing or package 15 having at least one reflective surface. The housing may be any appropriate container, for example, a case or a bag. In one embodiment, the housing may comprise a reflective bag, for example, a MYLAR® bag. The housing or package simultaneously creates homogeneous illumination and serves as a lightweight decontamination and storage container.

The plurality of violet-blue LEDs generate heat during sterilization. Thus, in embodiments, the sterilization device may comprise at least one fan and at least one air vent to exhaust heated air in the housing to keep the internal housing temperature at a desired level. FIG. 1 shows fan cover 20 and air vents 25. In a specific embodiment, the device may also include a control panel comprising at least one of display screen 30, mode selector 35, or on/off switch 40. The control panel may display at least one of temperature in the housing or time of sterilization; allow on/off device operation and/or fan operation; and allow a mode selection based upon available power source, for example, a battery or an external power source.

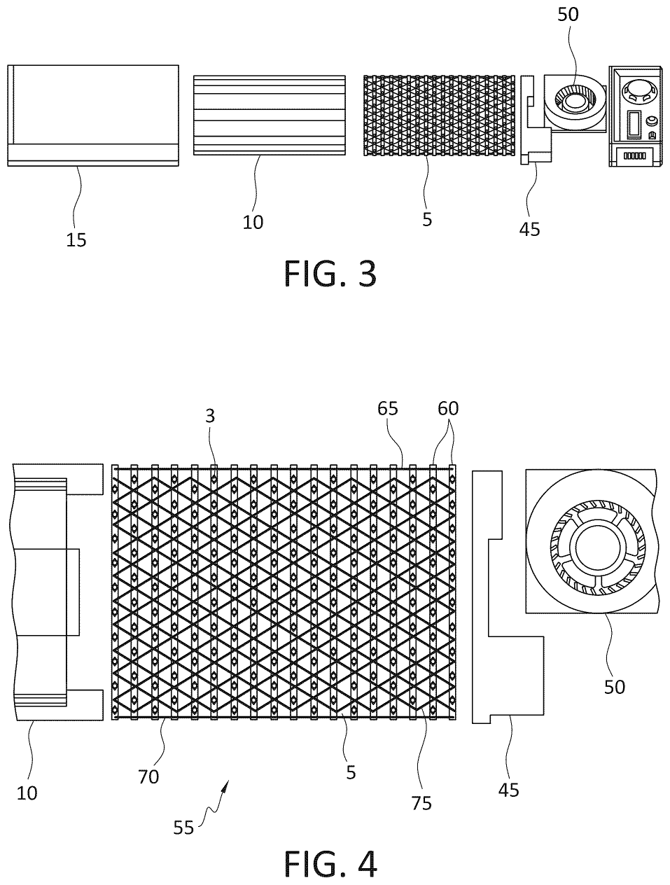

As shown in FIGS. 2-3, the device may have a modular structure in which the array 5 of violet-blue LEDs and/or flexible sheet 10 are connected to or removably attachable from a connector 45, which along with fan 50 are insertable into the control panel. The array 5/flexible sheet 10/and connector 45 can be placed in the housing 15.

As shown in FIGS. 2-3, but in greater detail in FIG. 4, the array 5 of violet-blue LEDs 3 may be mounted on a circuit board structure 55. The circuit board structure 55 may comprise a plurality of metal strips 60 (e.g., made from aluminum, copper, and the like), for example about 5 to about 20 strips, on which one or more LEDs are mounted or attached. In a specific embodiment, there may be about 2 to about 10 LEDs per strip, for example 5 to 10 LEDs per strip. The metal strips are each connected to a positive voltage supply rail 65 and a negative ground power rail 70.

In embodiments, the plurality of metal strips 60 may be connected by a wire mesh 75, which may be at least partially or fully embedded in the flexible sheet 10. The wire mesh 75 provides strength to the array 5 and helps prevent and kinks or breaks in the flexible sheet 10. The wire mesh 75 may be electrical wire, for example 20 gauge electrical wire, without any current.

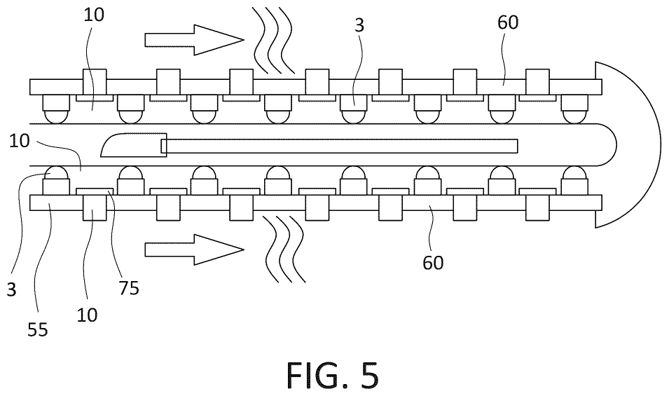

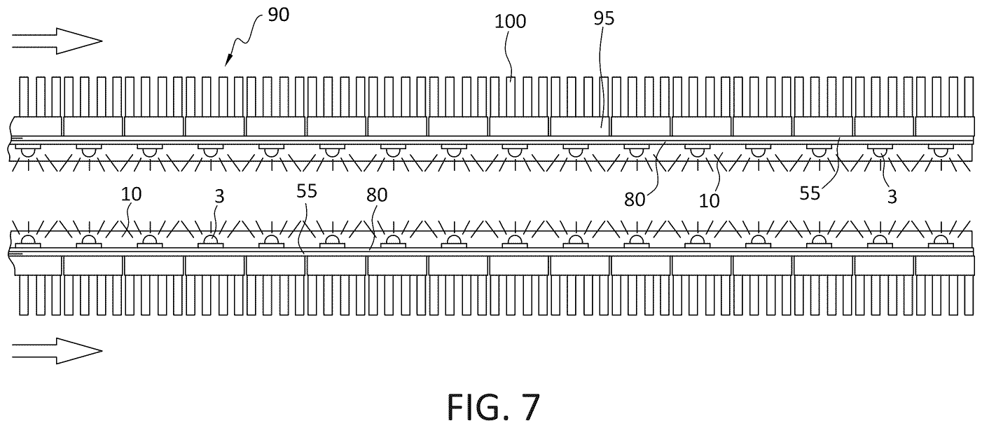

As shown in FIG. 5, the plurality of metal strips is configured so that a back or rear surface of the strips is exposed. In embodiments, the circuit board structure 55/array 5/wire mesh 75/flexible sheet 10 may be foldable into two or more layers before being placed into the housing (FIG. 5) so that the LEDs 3 face each other and a surgical device or tool may be inserted therebetween. In a specific embodiment, the circuit board structure 55/array 5/wire mesh 75/flexible sheet 10 may be rolled into a cylindrical shape. In other embodiments, the circuit board structure/array/flexible sheet, or any combination thereof, may be two or more distinct layers (FIG. 7). Heat (thermal energy) generated by the array of violet-blue LEDs may be transferred to the metal strips of the circuit board structure. In embodiments, the at least one fan may blow air over the exposed back or rear surface of the circuit board structure, so that the heated internal air of the device is vented from the device. Thus, the circuit board structure may act as a heat sink.

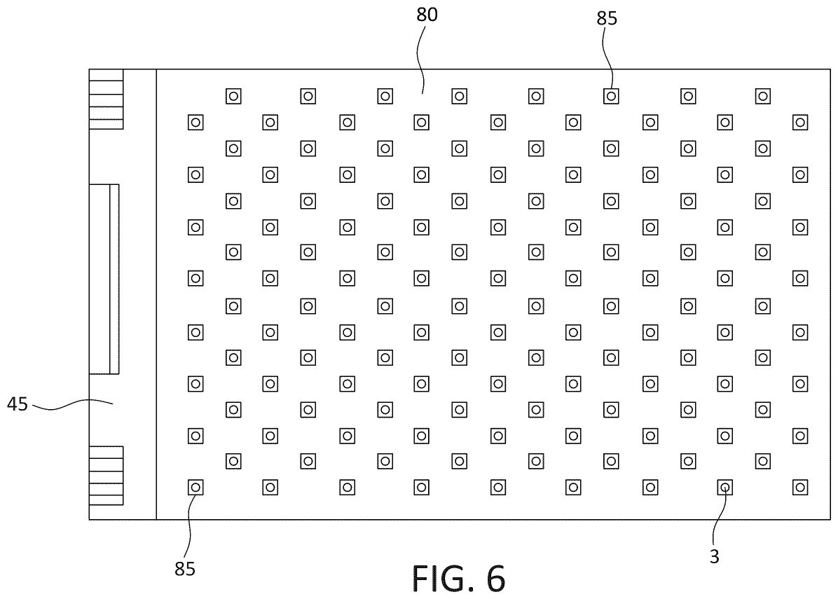

As shown in FIG. 6, in an embodiment, the flexible sheet 10 may have a reflective or mirrored sheet or coating 80. In embodiments, the reflective or mirrored sheet or coating 80 may be fully embedded within the flexible sheet 10. In a specific embodiment, the reflective or mirrored sheet or coating 80 may comprise a polyester-based film, for example 3M™ Reflective Polarizing Mirror (RPM). In embodiments, the flexible sheet may have a plurality of elements 85, with one violet-blue LED 3 mounted on and/or attached to each element 85. The shape of the elements is not necessarily a square, but may be any shape that allows fixation of an LED. The elements may comprise a plastic, metal, or ceramic. In an embodiment, a primer or adhesive may be used to fix the LEDs 3 to the sheet or coating 80 and/or to the elements 85. In a specific embodiment, there may be no metal strips 60 and/or no wire mesh 75.

As shown in FIG. 7, the sterilization device may comprise at least two separate layers, each layer positioned so that the respective flexible sheet 10 and LEDs 3 face each other and a surgical device or tool may be inserted therebetween. In an embodiment, each LED 3 has its own heat sink 90, located beneath the reflective or mirrored sheet or coating 80 and the circuit board structure 55. In an embodiment, the circuit board structure may comprise a flexible copper circuit configured to handle a current draw to power the LEDs. In embodiments, the heat sink 90 may comprise a body 95 having at least one, for example about two or more, projections or fins 100 extending outwardly therefrom and which aid in removal of thermal heat as air from the fan blows across each layer. In a specific embodiment, the heat sink is not connected to and/or surrounded by an insulating material. The heat sink 90 may comprise a lightweight material, such as aluminum or an aluminum alloy. The silicone sheets may help insulate a surgical device or tool from heat.

Irradiance from the array of violet-blue LEDs and the resulting temperature within the device during sterilization may be based on the number of LEDs. The time for sterilization may be based on the power source used. In specific embodiments, using a higher irradiance and more power may be beneficial for a faster sterilization. Radiant exposure may be about 180 to about 300 J/cm² and the irradiance may be about 50 mW/cm² or above.

A method for sterilizing an object according to the present invention comprises placing or wrapping an object in need of sterilization in the sterilization device; and applying violet-blue visible light from the violet-blue visible light LEDs to the object for a set amount of time, thereby sterilizing the object by reducing or eliminating at least one of bacteria, virus, fungus, yeast, or any combination thereof, on the object.

In embodiments, the object in need of sterilization may include, but is not limited to at least one medical or surgical instrument.

The invention may be illustrated by the following non-limiting example.

### Example

Irradiance and temperature during prototype development were measured using an LS133 UV LED energy meter and temperature probe. Radiant exposure (dosing) was calculated using the equation “Radiant exposure (J/cm²)=Irradiance (W/cm²)×Exposure time (seconds)” with dosing adjusted based upon duration of exposure. In addition, the utility of LED-delivered 405 nm light was assessed for 1 h (270 J/cm²) with or without a disinfectant already in use by ground surgical teams to reduce or eliminate bacterial burden from intentionally contaminated stainless steel bars (2×1 cm).

After euthanasia of 15 mice on an unrelated IACUC-approved protocol, the abdominal cavity was entered, and autoclaved metal bars were intentionally contaminated with cecal contents and blood mimicking a traumatic abdominal injury (4 bars/animal). Treatments (n=12 bars/group) included:
Group 1 (uncontaminated/negative control);
Group 2 (contaminated/no cleaning/positive control);
Group 3 (contaminated/water-soaked gauze to remove visible bioburden);
Group 4 (contaminated/water-soaked gauze/CaviCide disinfectant);
Group 5 (contaminated/water-soaked gauze/405 nm light); and Group 6 (contaminated/water-soaked gauze/CaviCide/405 nm light).

Antibacterial efficacy was determined by evaluation of viable bacteria recovered from specimens after swabbing and culturing on blood agar plates for 24 to 48 hours at 37° C.

Initial prototype design using an array of silicone-embedded LED strips resulted in an average irradiance of 10 mW/cm² which was insufficient to achieve the targeted minimum radiant energy dose of 100-200 J/cm² after 1 hour exposure. Redesign using more powerful 405 nm LEDs and a micro-cooling fan increased irradiance values above 50 mW/cm².

Violet-blue light irradiation (Group 5) resulted in 0/12 (0%) samples having recoverable bacterial growth (similar to CaviCide), while cleaning with water-soaked gauze alone (Group 3) resulted in considerable bacterial growth (12/12 or 100% positive samples). Therefore, 405 nm light delivered for 1 h (270 J/cm²) could achieve a high level of disinfection.

### INDUSTRIAL APPLICABILITY

The present invention is directed to a sterilization device having a plurality of violet-blue visible light emitting diodes (LED) at least partially or fully embedded in or attached to at least one flexible sheet and contained in a housing having at least one reflective surface.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. A sterilization device, comprising:
at least one flexible sheet;
a plurality of violet-blue visible light light-emitting diodes (LEDs), wherein each LED is fully embedded in the at least one flexible sheet; and
a housing having at least one reflective surface into which the at least one flexible sheet is insertable.

2. The sterilization device according to claim 1, comprising an array of violet-blue LEDs having a wavelength of about 400 to about 420 nm.

3. The sterilization device according to claim 1, comprising an array of violet-blue LEDs having a wavelength of about 405 nm.

4. The sterilization device according to claim 1, wherein the at least one flexible sheet comprises a silicone sheet.

5. The sterilization device according to claim 4, wherein the silicone sheet has a thickness of about 0.1 to about 1 mm.

6. The sterilization device according to claim 1, wherein the housing comprises a MYLAR® bag.

7. The sterilization device according to claim 1, further comprising a circuit board structure comprising a plurality of metal strips, each strip attached at one end to a positive voltage supply rail and at an opposing end to negative ground power rail, wherein two or more violet-blue LEDs are affixed or attached to each metal strip.

8. The sterilization device according to claim 7, wherein the metal strips comprise aluminum.

9. The sterilization device according to claim 7, wherein the plurality of metal strips is configured so that a back or rear surface of each strip is exposed to the air within the sterilization device.

10. The sterilization device according to claim 1, wherein the violet-blue LEDs are connected via a wire mesh.

11. The sterilization device according to claim 1, further comprising at least one fan and at least one air vent.

12. The sterilization device according to claim 1, further comprising at least one rechargeable battery.

13. The sterilization device according to claim 1, wherein the device weights about 1 pound to about 5 pounds.

14. The sterilization device according to claim 1, wherein the at least one flexible sheet comprising the plurality of violet-blue visible light LEDs is folded into two layers and is configured so that a number of LEDs face each other and a space is formed between the two layers for an object to be sterilized.

15. The sterilization device according to claim 1, further comprising a reflective or mirrored sheet or coating embedded in the at least one flexible sheet.

16. The sterilization device according to claim 1, wherein the at least one flexible sheet has a plurality of elements, with one violet-blue LED mounted on and/or attached to each element.

17. The sterilization device according to claim 1, wherein each LED has its own heat sink comprising a body having at least one projection or fin extending outwardly from the body.

18. The sterilization device according to claim 17, wherein the heat sink comprises two or more projections or fins extending outwardly from the body.

19. The sterilization device according to claim 17, comprising at least two separate layers, each layer comprising at least one flexible sheet and a plurality of violet-blue visible light LEDs, wherein the at least two separate sheets are configured so that the respective LEDs face each other and a space is formed between the at least two separate layers for an object to be sterilized.

20. A method for sterilizing an object, comprising:
placing or wrapping an object in need of sterilization in the device according to claim 1; and
applying violet-blue visible light from the violet-blue visible light LEDs to the object for a set amount of time, thereby sterilizing the object by reducing or eliminating bacteria or viruses on the object,
wherein the object in need of sterilization comprises at least one surgical instrument, wherein the radiant exposure is about 180 J/cm² to about 300 J/cm².
